(19) United States
(12) Patent Application Publication
(10) Pub. No.: US 2011/0281804 A1
(43) Pub. Date: Nov. 17, 2011

(54) PEPTIDIC AND PEPTIDOMIMETIC COMPOUNDS FOR REGULATING AUTOPHAGY

(75) Inventors: Maria Paola Paronetto, Roma (IT); Gian Maria Fimia, Grottaferrata (IT); Sabrina Di Bartolomeo, Roma (IT)

(73) Assignee: FONDAZIONE SANTA LUCIA, Roma (IT)

(21) Appl. No.: 13/058,644

(22) PCT Filed: Aug. 11, 2009
(86) PCT No.: PCT/EP09/60399

§ 371 Date: Jul. 28, 2011

(30) Foreign Application Priority Data
Aug. 12, 2008 (EP) 08162269.8

(51) Int. Cl. A61K 38/08; A61K 38/10; A61P 35/00; A61P 25/14; C07K 7/06; A61P 25/28

(52) U.S. Cl. 514/17.8; 530/328; 514/21.7; 530/327; 530/326; 514/17.7; 514/19.3

(57) ABSTRACT

The present invention relates to peptides, peptidomimetic compounds and pharmaceutical uses thereof for the treatment of neurodegenerative diseases or tumourigenesis and more specifically diseases deriving from the dysregulation of the signalling system of Ambra-1-mediated autophagy.

### TECHNICAL FIELD

The present invention relates to peptides, peptidomimetic compounds and pharmaceutical uses thereof for the treatment of neurodegenerative diseases or tumourigenesis and more specifically diseases deriving from the dysregulation of the signalling system of Ambra-1-mediated autophagy.

### STATE OF THE ART

Autophagy is a catabolic process involving the degradation of a cell's own components through the lysosomal machinery. It is a tightly-regulated process that plays a normal part in cell growth, development, and homeostasis, helping to maintain a balance between the synthesis, degradation, and subsequent recycling of cellular products. It is a major mechanism by which a starving cell reallocates nutrients from unnecessary processes to more-essential processes.

Autophagy can help to profoundly and rapidly renovate cells or modify their external appearance within a few hours. As expected, any genetic or pharmacological alteration in this process impairs cell survival rate or cell metabolism, thereby affecting tissue homeostasis (Levine and Kroemer, 2008; Mizushima et al., 2008).

Many neurodegenerative conditions can be traced back to defective autophagy, which may be the cause of the failure to clear aggregates of mutated toxic proteins (Rubinsztein, 2006). Autophagy has also been identified as a crucial process in oncogenesis and cancer progression (Jin and White, 2007; Levine and Kroemer, 2008; Mizushima et al., 2008). Several autophagy-related proteins have tumour suppressor activity (Beclin 1, Atg5, Bif-1, Atg4C, UVRAG) and some autophagy gene mutations can lead to an accumulation of DNA damage and genome instability (Mathew et al., 2007). Finally, antigen presentation, innate immune signalling and pathogen degradation may all involve autophagosome recruitment and activity; therefore, autophagy genes may play an important role in immunity and infectious diseases (Levine and Deretic, 2007).

A blockade in maturation and/or an upregulation of autophagy has/have been shown to occur in neurodegenerative conditions. Degeneration and neuronal death may occur when this autophagic response becomes overwhelmed by the extent of the cellular damage and as a consequence loses its neuroprotective role. Remarkably, in the case of AD, autophagy could also promote the build up of toxic amyloid-β (Aβ) peptide by providing a suitable environment for Aβ synthesis and storage. The accumulation of Aβ in the autophagosome can in turn destabilise its membrane and halt the degradation process. In this light, autophagic failure rather than autophagic upregulation seems one of the possible keys to interpret the pathogenesis of AD, Huntington's disease (HD), Batten disease and many other neurodegenerative disorders.

Neurodegenerative disorders are increasingly prevalent diseases in the Western world, they are dramatically debilitating, often display a long and progressive decline and eventually result in death. The management of patients affected by these diseases represents a very heavy burden for public health costs. Unfortunately, the mechanisms underlying most of these diseases is still to be unravelled and few therapeutic compounds are known for the effective treatment of the same.

As there is currently no cure for most of these diseases and their treatment only focuses on the management of symptoms and is still scarcely effective, the need is felt to find medicaments for the treatment of these disorders.

### SUMMARY OF THE INVENTION

It is the object of the present invention to provide a peptide comprising a TQT amino acid triplet followed by at least 5 amino acid residues forming an α-helix secondary structure.

The present invention satisfies the above identified needs by providing a peptide as defined in claim 1.

It is another object of the present invention to provide a peptidomimetic compound comprising at least one portion having the same 3D conformation of a peptide according to claim 1.

It is another object of the present invention to provide a pharmaceutical composition comprising at least one peptide according to claim 1 or peptidomimetic compound according to claim 5 in mixtures with at least one pharmaceutically acceptable vehicle and/or excipient.

It is a further object of the present invention to provide a pharmaceutical composition comprising at least one peptide according to claim 1 or peptidomimetic compound according to claim 5 or pharmaceutical composition according to claim 7 for preventing the binding of Ambra1 to DLC1 by interfering with their reciprocal interaction and for the treatment of diseases deriving from the dysregulation of the signalling system of Ambra1-mediated autophagy.

### Definitions

As used herein, the term “peptidomimetic compound” refers to a synthetic molecule that resembles in structure and steric conformation that of the peptide after which has been designed. In particular, these peptidomimetic compounds are assembled with modified amino acids and/or organic molecules that have the same 3D conformation of the L-amino acid but are not recognized by cellular and extra-cellular proteases. A similar approach has been recently validated by a synthetic molecule mimicking the structure of a MyD88 inhibitory peptide. Remarkably, this peptide-mimetic compound (ST2825) maintained the same activity and specificity of inhibition displayed by the original MyD88 inhibitory peptide, proving the feasibility of this approach (Loiarro et al., J. Leukocyte Biology 2007).

As used herein, the term “AMBRA1” refers to the protein disclosed in Fimia G. M et al. Nature 447 (7148), 1121-1125 (2007) (accession number ABI74670 GI: 114432124).

### BRIEF DESCRIPTION OF THE DRAWINGS

The present invention will now be described with reference to the accompanying drawings, wherein:

FIG. 1: DLC1 is an AMBRA1-interacting protein

(a), AMBRA1-DLC1 interaction in mammalian cells. 2F cells were infected with a retroviral vector encoding HA-DLC1 or an empty vector (Ctr). Protein extracts were immunoprecipitated using an anti-HA antibody [IP HA (DLC1)]. Purified complexes and corresponding total extracts were analysed by western blot using anti-AMBRA1 (WB AMBRA1, left panels) or anti-HA antibodies (WB HA, right panels). (b), Ambra1-Dlc1 interaction in mouse embryos. Protein extracts from embryos at developmental day 14.5 wild-type (+/+), heterozygous (+/gt) and homozygous (gt/gt) for the Ambra1 gene trap mutation were immunoprecipitated using an anti-AMBRA1 antibody (IP Ambra1). Purified complexes and corresponding total extracts were analysed by western blot using an anti-DLC1 antibody (WB Dlc1). (c-d), AMBRA1-DLC1 co-localisation in mammalian cells. (c), Confocal analysis of 2F cells co-expressing HA-DLC1 and AMBRA1 stained by anti-HA and anti-AMBRA1 antibodies. The merge image is shown in the right panel. Scale bar, 20 μm. (d), Immunogold analysis of 2F cells co-expressing HA-DLC1 and AMBRA1. 15 nm and 5 nm gold particles label the AMBRA1 and DLC1 protein, respectively. Black arrows point to AMBRA1-DLC1 co-localising proteins, while grey arrows indicate a single DLC1 molecule. Scale bar: 45 nm. (e-f), Characterisation of the DLC1 interacting domain of AMBRA1. (e), 2F cells were co-infected with retroviral vectors encoding HA-tagged DLC1 and the indicated myc-tagged AMBRA1 proteins or myc-tagged β-galactosidase (βgal) as a negative control. A scheme of the AMBRA1 mutants with the corresponding aminoacid sequence boundary is reported. FL, full length; F1, F2, F3, Fragments 1-3. Protein extracts were immunoprecipitated using an anti-myc antibody (IP myc). Purified complexes and corresponding total extracts were analysed by western blot using anti-myc (WB myc, upper panels) and anti-HA antibody [WB HA (DLC1), lower panels]. (f), 2F cells were co-infected with retroviral vectors encoding HA-tagged DLC1 and the myc-tagged AMBRA1 FL or AMBRA1 mutants TAT1 and TAT2. The position within the AMBRA1 sequence of the two TQT domains (1 and 2), which were mutated in the TAT mutants, is shown (see box). Protein extracts were immunoprecipitated using an anti-myc antibody (IP myc). Purified complexes and corresponding total extracts were analysed by western blot using an anti-myc (WB myc, upper panels) and anti-HA antibody [WB HA (DLC1), lower panels].

FIG. 2. Modulation of AMBRA1-dynein interaction during autophagy

(a), AMBRA1-DLC1 interaction upon autophagy induction. 2F cells co-infected with retroviral vectors encoding HA-tagged DLC1 and AMBRA1 proteins were nutrient-starved for 4 hrs (+) or left untreated (−). Protein extracts were immunoprecipitated with an anti-HA tagged antibody [IP HA (DLC1)] or, as a negative control, with an unrelated antibody (IP Ctr). Purified complexes were analysed together with the corresponding total extracts by western blotting using anti-AMBRA1 (WB AMBRA1, upper panels) and anti-DLC1 antibodies (WB DLC1, lower panels). (b), AMBRA1 dissociates from the dynein motor complex during autophagy. 2F cells were nutrient-starved for 4 hrs or left untreated. Protein extracts were immunoprecipitated using an anti-DIC (IP DIC) or an unrelated antibody (IP Ctr) as a negative control. The purified complexes were analysed by western blotting together with the corresponding total extracts by using anti-DIC (WB DIC, upper panels), anti-AMBRA1 (WB AMBRA1, middle panels) and anti-DLC1 antibodies (WB DLC1, lower panels). (c), Ambra1 dissociates from the dynein motor complex in mouse tissues following autophagy induction. Mice from the same litter were kept without food for 24 and 48 hrs or fed ad libitum (−) before sacrifice and necropsy. Kidneys from these mice were homogenised and subjected to immunoprecipitation analysis by using an anti-Dic antibody (IP Dic) or with an unrelated antibody (IP Ctr). Protein immunocomplexes were then probed together with the corresponding total extracts using anti-Dic (WB Dic, upper panels), anti-Ambra1 (WB Ambra1, middle panels) and anti-Dlc1 (WB Dlc1, lower panels) antibodies. (d), AMBRA1-Tubulin interaction upon autophagy induction. 2F cells infected with retroviral vectors encoding βgal or AMBRA1 FLAG proteins were nutrient-starved for 4 hrs or left untreated. Protein extracts were then immunoprecipitated with an anti-FLAG antibody (IP FLAG). Purified proteins were eluted using the FLAG peptide and analysed by western blotting together with the corresponding total extracts using anti-AMBRA1 (WB AMBRA1, upper panels) and anti-Tubulin antibodies (WB Tubulin, lower panels). (e), Down-regulation of ULK1 expression prevents AMBRA1 dissociation from the dynein motor complex during autophagy. 2F cells were transfected using ULK1 siRNA oligos (siULK1) or unrelated siRNA oligos (siCtr). Forty eight hrs after transfection, the cells were nutrient-starved for 4 hrs or left untreated. Protein extracts were immunoprecipitated using an anti-DIC (IP DIC) or an unrelated antibody (IP Ctr) as a negative control. The purified complexes were analysed by western blotting together with the corresponding total extracts using anti-DIC (WB DIC, lower panels) and anti-AMBRA1 antibodies (WB AMBRA1, upper panels). (f-g), ULK1 immunopurified complexes phosphorylate AMBRA1 in vitro. HEK293 cells were transfected with expression vectors encoding AMBRA1 or ULK1 myc tagged proteins [Wild type (Wt) or K46I ‘kinase-dead’ mutant]. After 24 hrs, ULK1-transfected cells were nutrient-starved for 2 hrs. Protein extracts were prepared from all transfected cells and immunoprecipitated using the anti-myc antibody (IP myc). A small aliquot of the immunoprecipitated proteins were analysed by western blotting using an anti-myc antibody (WB myc) to check for protein purification (f). Immunopurified ULK1 was subjected to an in vitro kinase assay in the presence or absence of immunopurified AMBRA1 as described in the ‘Methods’ section. A myc-tag immunoprecipitation on untransfected cells was used as a negative control (−). The reactions were resolved on SDS-PAGE and 32P-labeled proteins revealed by autoradiography (g).

FIG. 3. Dynamics of AMBRA1 interaction with the BECLIN 1/VPS34 complex.

(a-b), AMBRA1 re-localises to ER upon autophagy induction. (a) 2F cells infected with retroviral vectors encoding AMBRA1 protein were starved for 4 hrs or left untreated, fixed and stained with the anti-AMBRA1 and anti-Erp57 antibodies. (b) 2F cells transiently co-transfected with expression vectors encoding mCherryAMBRA1 and GFPDFCP1 were starved for 4 hrs or left untreated, fixed and analysed by confocal microscopy. The images showing the merge of the two fluorescence signals are shown in the lower panels. Scale bar, 8 μm. (c), VPS34-AMBRA1 interaction upon autophagy induction. 2F cells infected with retroviral vectors encoding_gal myc or AMBRA1 myc proteins were nutrientstarved for 4 hrs or left untreated. Protein extracts were immunoprecipitated with an anti-myc antibody (IP myc). Purified complexes were analysed together with the corresponding total extracts by western blotting using an anti-VPS34 antibody (WB VPS34). (d), BECLIN 1 interacts with DLC1. 2F cells were transiently transfected with expression plasmids encoding βgal or FLAG-tagged BECLIN 1, and nutrient-starved for 4 hrs or left untreated. Protein extracts were immunoprecipitated with an anti-FLAG antibody. Purified complexes were analysed together with the corresponding total extracts by western blotting using anti-BECLIN1 (WB BECLIN 1, upper panels), anti-AMBRA1 (WB AMBRA1, middle panels) and anti-DLC1 antibodies (WB DLC1, lower panels). (e), Dynamic interaction of BECLIN 1 with the dynein motor complex during autophagy. Protein extracts from AMBRA1-overexpressing 2F cells were immunoprecipitated using an anti-DIC antibody (IP DIC) or an unrelated antibody (IP Ctr). The purified complexes were analysed together with the corresponding total extracts by western blotting using an anti-BECLIN 1 antibody (WB BECLIN 1). BECLIN 1-DIC interaction was also analysed in starved or untreated cells (right panels). (f-g) Co-localisation of AMBRA1 with BECLIN 1 and PI(3)P-enriched membranes upon autophagy induction. (f) 2F cells infected with retroviral vectors encoding AMBRA1 were starved for 4 hrs or left untreated, fixed and stained with anti-AMBRA1 and anti-BECLIN 1 antibodies. (g), 2F cells transiently transfected with mCherryAMBRA1 and GFP-p40Phox cDNAs were starved for 4 hrs or left untreated, fixed and analysed by confocal microscopy. The images showing the merge of the two fluorescence signals are shown in the lower panels. Scale bar, 20 μm.

FIG. 4. Modulation of autophagy by DLC1 down-regulation

(a), DLC1 down-regulation in GFP-LC3-expressing 2F cells using specific siRNA oligonucleotides (siDLC1a and siDLC1b). DLC1 mRNA and protein levels were analysed by quantitative PCR (left) and Western blotting (right). siCtr, unrelated oligo. R.L., relative levels. Tubulin, protein loading control. (b), Increase of basal and starvation-induced autophagy by DLC1 down-regulation. 24 hrs after transfection with DLC1 siRNA oligonucleotides, GFP-LC3 expressing 2F cells were starved for 4 hrs or left untreated, and the occurrence of autophagy was analysed by measuring GFP-LC3-punctate positive cells. A graph reporting data from three experiments is shown together with representative fluorescence images of siRNA oligonucleotides-transfected cells in control conditions. Scale bar: 20 μm. (c), Ultrastructural analysis by means of electron microscopy of ultra-thin sections from 2F cells transfected with a RNAi oligo for DLC1 (siDLC1b) or an unrelated oligo (siCtr), and starved for 4 hrs or left untreated. Representative images are accompanied by a graph reporting the number of autophagosomes per field (48 square μm). Scale bar, 1.5 μm. (d-f) Increase of autophagosome on-rate by DLC1 down-regulation. (d), After DLC1 downregulation (siDLC1a-b), 2F cells were treated with the lysosome inhibitors E64d and Pepstatin A for 4 hrs or left untreated, and the occurrence of autophagy was analysed by LC3-I to LC3-II conversion. Densitometry analysis showing the band density ratio of LC3 II band relative to Tubulin is reported on the accompanying graph. (e), mCherryLC3-expressing 2F cells transfected as in (d) and treated with the lysosome inhibitors E64d and Pepstatin A for 4 hrs, were stained by using the lysosome marker LAMP1 (green). The images showing the merge of the two fluorescence signals are shown in the lower panels. An insert containing a higher magnification area of the merge images is also shown. Scale bar, 16 μm. (f), GFPLC3-expressing 2F cells, transfected and treated as in (d), were analysed for the appearance of GFP-LC3 puncta per cell. Values in (a-c) and (f) represent the mean±s.d. of three experiments. * P<0.05; **, P<0.01; *** P<0.001.

FIG. 5. DLC1 regulates AMBRA1 function.

(a), Autophagy induced by DLC1 down-regulation requires AMBRA1. GFP-LC3-expressing 2F cells were transfected using DLC1 and AMBRA1 siRNA oligonucletides (siAMBRA1) either separately or in combination. 24 hrs after transfection, 2F cells were starved for 4 hrs or left untreated and the occurrence of autophagy was analysed by measuring GFP-LC3 punctate positive cells. (b), Autophagy induced by DLC1 down-regulation requires PI3K activity. After DLC1 down-regulation, 2F cells were incubated with Wortmannin for 4 h and analysed for appearance of GFP-LC3 punctate staining. (c-d), AMBRA1 mutants defective for DLC1 interaction have an increased autophagic potential. GFP-LC3-expressing 2F cells were transduced with retroviral vectors encoding AMBRA1 wild type (AMBRA1 FL) or TAT1 and TAT2 mutants (AMBRA1TAT1 and TAT2) and analysed for the appearance of GFPLC3 punctate staining (c) or for acidic vesicular organelle formation by FACS measurement of Acridine Orange staining (d). A βgal retroviral vector was used as a negative control. Scale bar, 20 μm. Values in (a-d) represent the mean±s.d. of three experiments.

### DETAILED DESCRIPTION OF THE INVENTION

According to the present invention a peptide comprising a TQT amino acid triplet followed by at least 5 amino acid residues forming an a-helix secondary structure is provided. The peptide preferably comprises SEQ ID NO: 1, SEQ ID NO: 2, or SEQ ID NO: 3. More preferably the peptide is SEQ ID NO: 1, SEQ ID NO: 2, or SEQ ID NO: 3.

According to the present invention a peptidomimetic compound is also provided comprising at least one portion having the same 3D conformation as the peptide defined above.

In particular, the peptide or peptidomimetic compound can be used as a medicament.

Further, according to the present invention a pharmaceutical composition is provided comprising at least one peptide or peptidomimetic compound as defined above in mixtures with at least one pharmaceutically acceptable vehicle and/or excipient.

The peptide, peptidomimetic compound or composition as defined above can be used for preventing the binding of Ambra1 to DLC1 by interfering with their reciprocal interaction, in particular for the treatment of diseases deriving from the dysregulation of the signalling system of Ambra1-mediated autophagy, such as neurodegenerative diseases or tumourigenesis.

The neurodegenerative disease is advantageously Alzheimer's disease, Huntington's disease and Batten disease.

Autophagy is a cellular process mediating degradation of bulk cytoplasm, long-lived proteins and entire organelles. In this process, double-membraned vesicles, termed autophagosomes, wrap around portions of cytosol and transport them to the lysosome for degradation. Several molecules participate in autophagosome nucleation and elongation, including various components of the class III phosphatidylinositol-3-OH kinase (PI3K) complex (Ambra1, Atg14L/Barkor, Beclin 1, Rubicon, UVRAG, Vps34, Vps15) and most of the Atg genes. The autophagosome movement towards the lysosome is dependent on microtubules and the dynein motor complex. Besides its role as a cytoskeletal motor, the dynein complex is also a docking system for regulatory factors involved in a number of signalling pathways. In particular, the dynein light chains DLC1 and DLC2 are involved in cell death regulation by sequestering pro-apoptotic proteins.

In the following, autophagosome formation in mammalian cells is shown to be primed by Ambra1 release from the dynein motor complex. It has been found that Ambra1 specifically binds the dynein motor complex under normal conditions through a direct interaction with DLC1. When autophagy is induced, Ambra1-DLC1 are released from the dynein complex in an ULK1-dependent manner, and relocalise to the endoplasmic reticulum, thus enabling autophagosome nucleation. In addition, it has been found that both DLC1 down-regulation and Ambra1 mutation in its DLC1-binding site strongly enhance autophagosome formation. Ambra1 is therefore not only a co-factor of Beclin 1 in favouring its kinase-associated activity but also a crucial upstream regulator of autophagy initiation. These results demonstrate that, besides microtubule's role in mediating autophagosome transport within the cell, there is a strict and regulative relationship between cytoskeleton dynamics and autophagosome formation. Unravelling the mechanism of autophagy initiation in higher eukaryotes will be relevant for designing new molecules for interfering with this process.

Ambra1 has been identified as a crucial factor in regulating autophagy in vertebrates. Its inactivation in vivo gives rise to defects in the developing nervous system and to embryonic death. Ambra1 promotes Beclin 1 interaction with its target lipid kinase Vps34, the core of a signalling complex that mediates autophagosome nucleation.

AMBRA1 central domains are required for its interaction with BECLIN 1. To further dissect the molecular mechanisms of AMBRA1 function, a yeast two-hybrid assay was performed using a cDNA encoding the C-terminal 533-1269 amino acids of the human AMBRA1 protein. By screening a human brain cDNA library, the dynein light chain 1 protein (DLC1, also known as DYNLL1, LC8, and PIN) was isolated as an AMBRA1 interactor (data not shown).

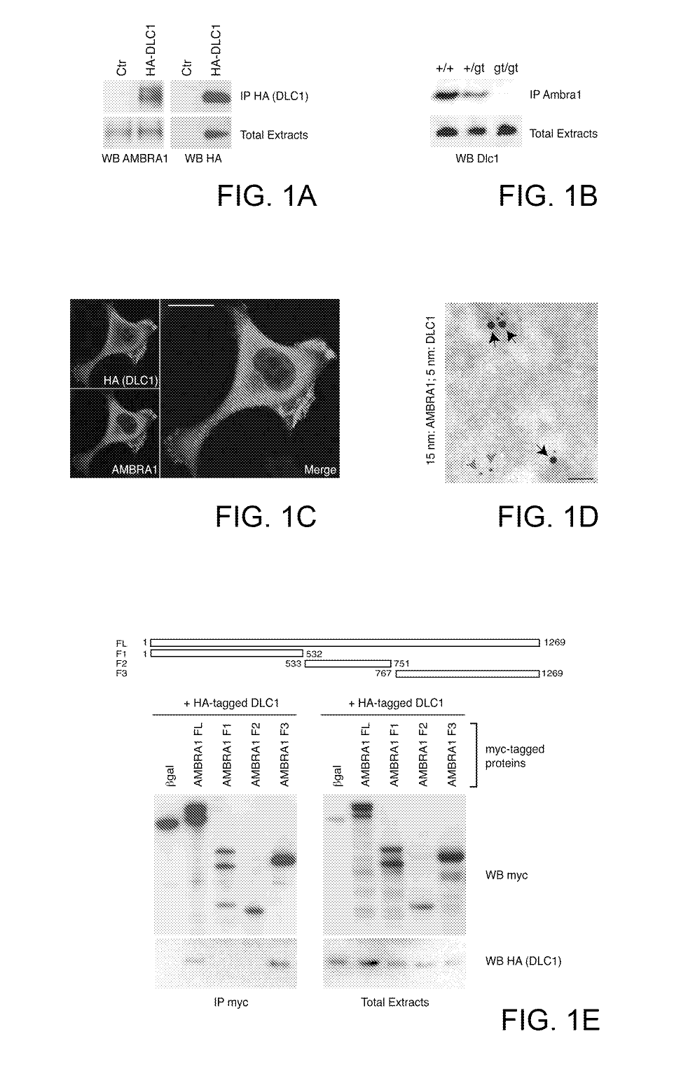

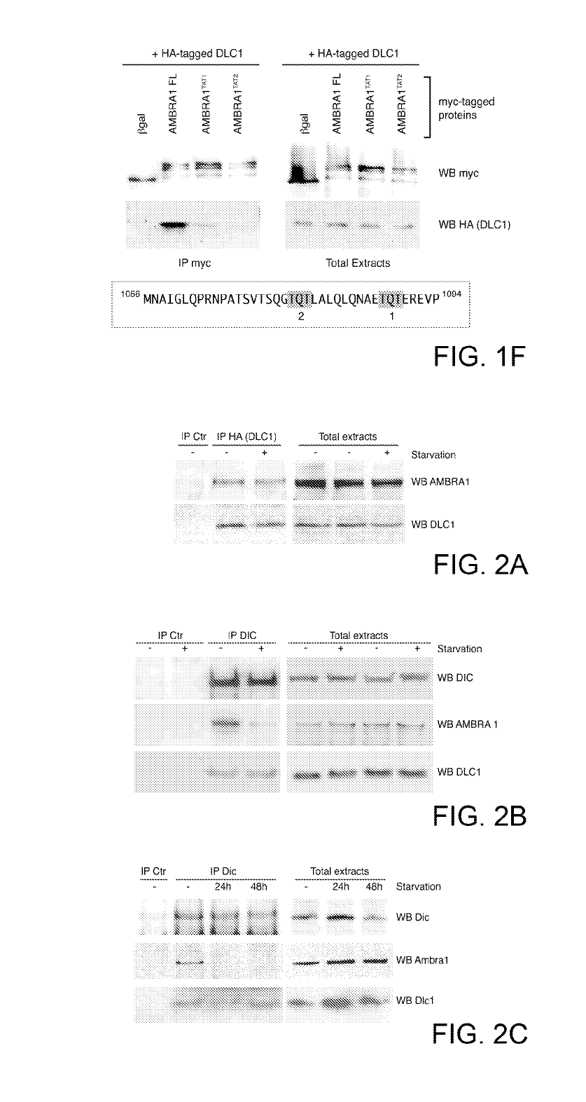

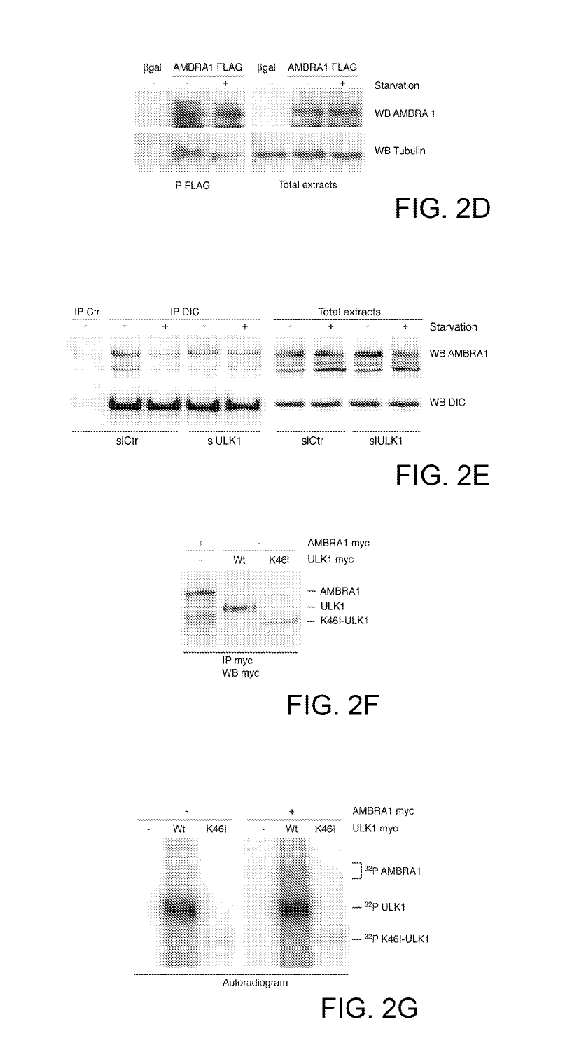

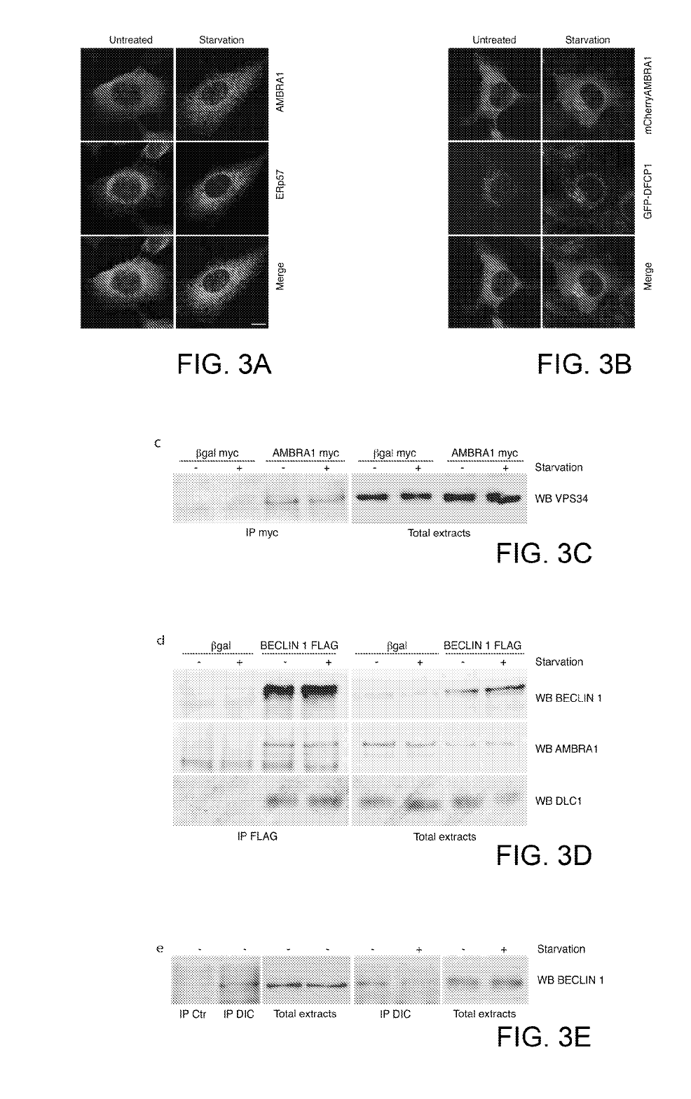

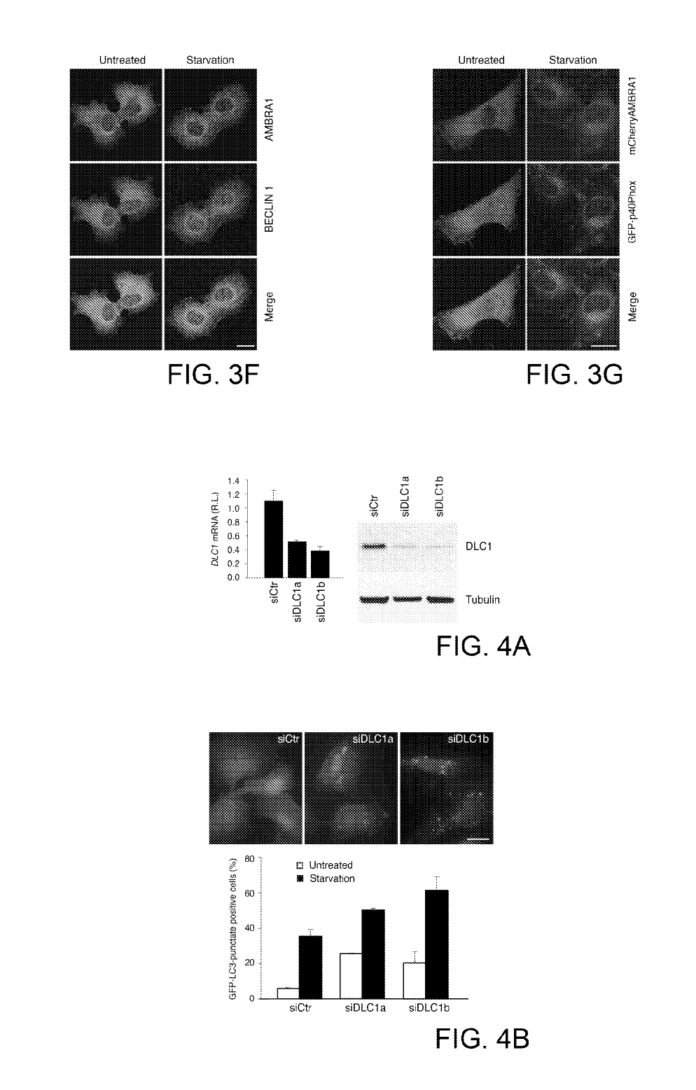

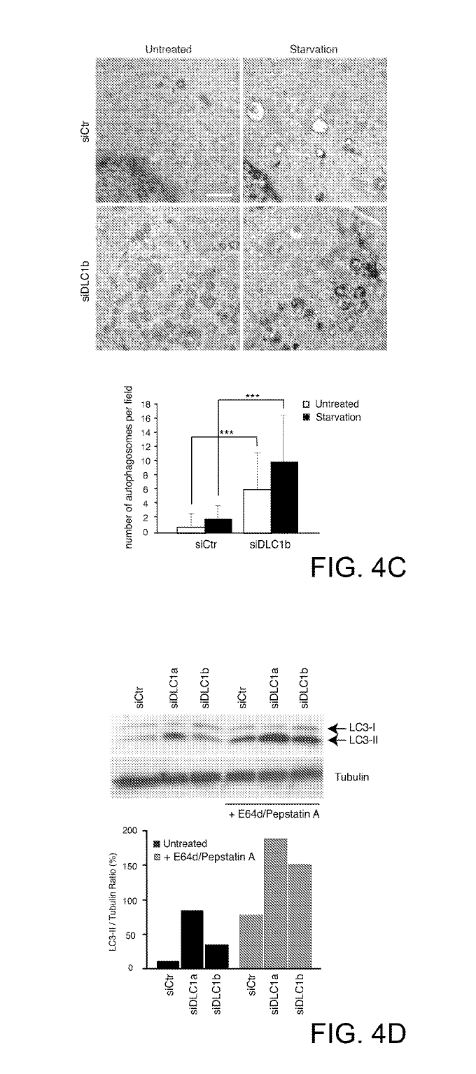

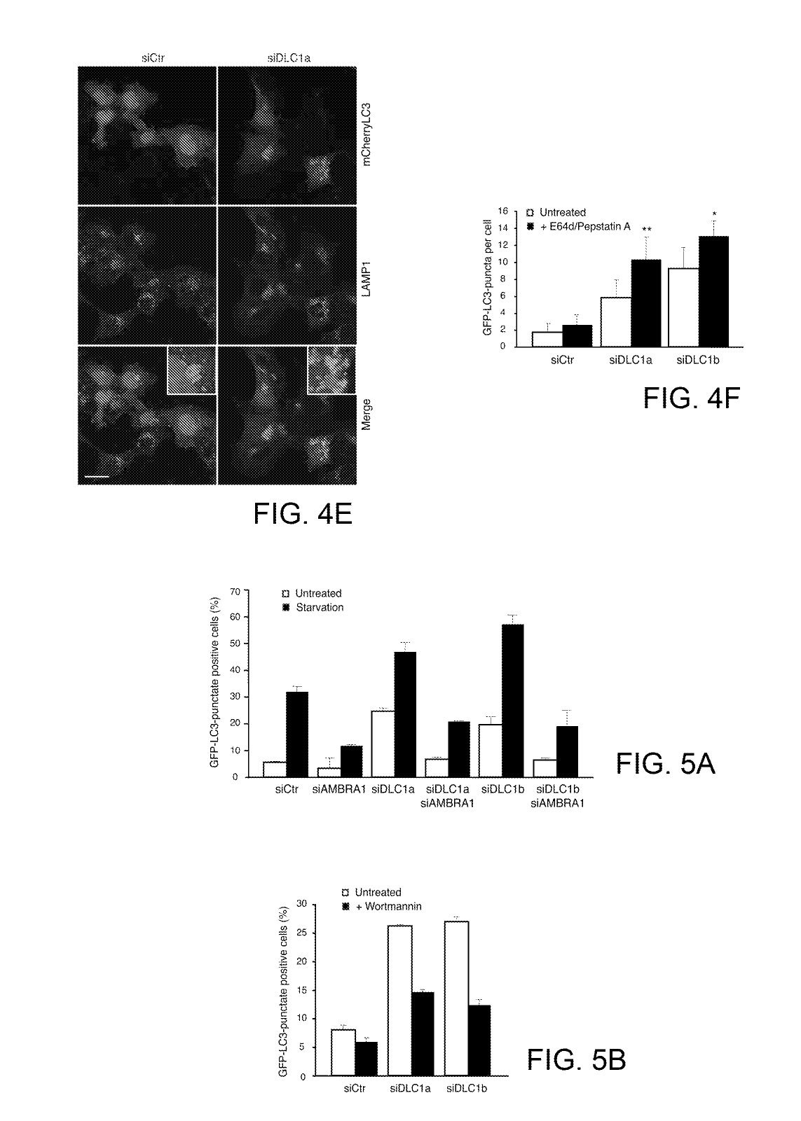

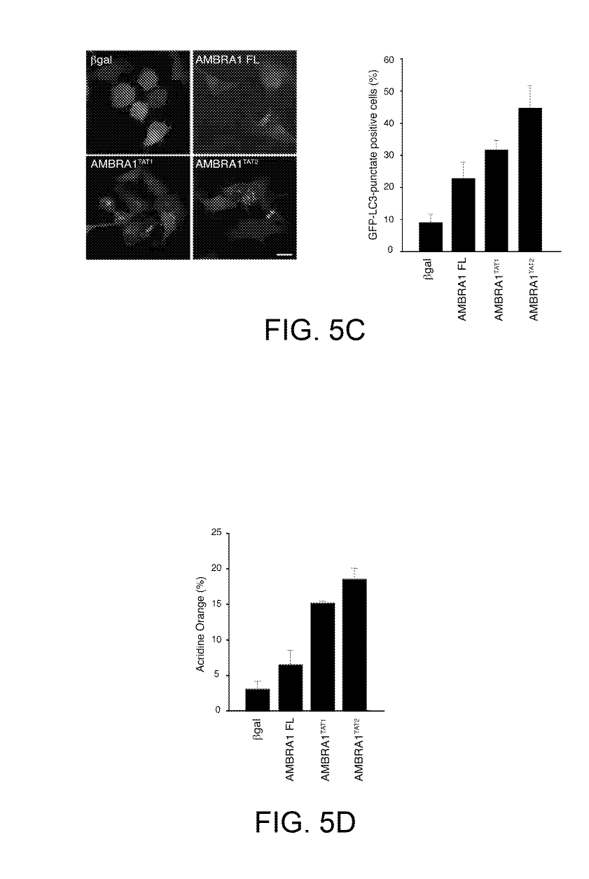

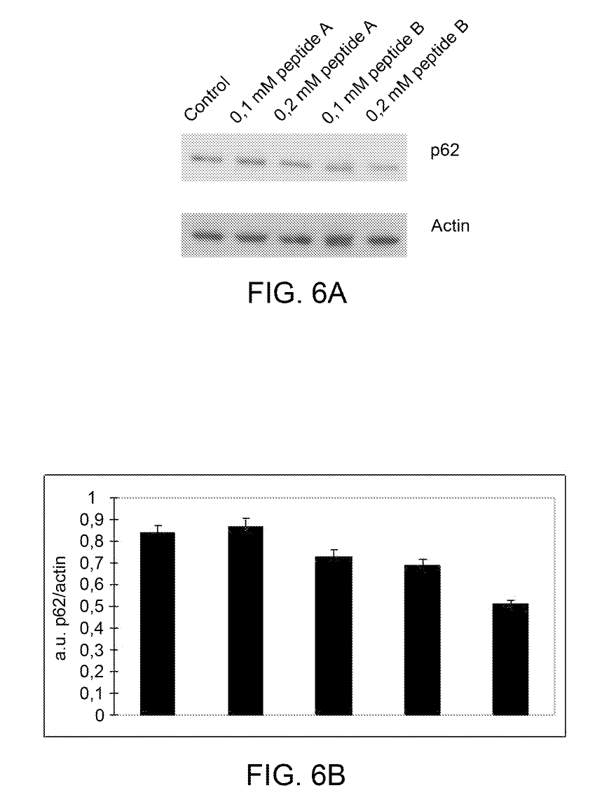

First, AMBRA1-DLC1 interaction was confirmed in vivo by a coimmunoprecipitation assay. DLC1 and AMBRA1 associated effectively with each other when co-expressed in human 2FTGH (2F) fibroblasts (FIG. 1a). Their binding was also tested in embryonic tissues, using protein extracts prepared from wild-type (wt) and Ambra1-mutant (Ambra1^{gt}) embryos. Dlc1 co-purified with Ambra1 in wt and Ambra1^{+/gt} samples, but not in samples where both Ambra1 alleles are mutated (Ambra1^{gt/gt}) (FIG. 1b). Consistent with their interaction in vivo, DLC1 and AMBRA1 showed a significant colocalisation by both confocal immunofluorescence and immuno-gold assays (FIG. 1c, d).

In order to map the AMBRA1 region responsible for DLC1 binding, different AMBRA1 cDNA deletion constructs were transduced in 2F cells and tested for their capacity to coimmunoprecipitate with DLC1. As shown in FIG. 1e, the AMBRA1 F3 fragment (C-term) is sufficient to bind DLC1, whereas the AMBRA1 N-terminal (F1) and central region (F2) show no interaction. By in silico analysis, two putative DLC1-binding motifs (TQT) on the AMBRA1 C-terminal sequence were then identified. To verify whether these motifs were indeed required for interaction, two mutated cDNA constructs were generated, namely AMBRA1^{TAT1} and AMBRA1^{TAT2}, carrying a Q¹⁰⁸⁸→A and a Q¹⁰⁷⁶→A point-mutation within the putative binding sites 1 and 2, respectively. By co-immunoprecipitation assays both AMBRA1^{TAT1} and AMBRA1^{TAT2} were observed to interact with DLC1 to a very low extent compared to the AMBRA1 wt (FL) protein (FIG. 1f).

Due to the important role played by AMBRA1 in regulating autophagy, its interaction with DLC1 was analysed upon autophagy induction. 2F cells co-expressing AMBRA1 and DLC1 were incubated in a nutrient-free medium for 4 hours and were then analysed by reciprocal co-immunoprecipitation assays. As shown in FIG. 2a, AMBRA1-DLC1 interaction is not altered by autophagy stimulation. Accordingly, AMBRA1 shows a significant co-localisation with DLC1 in 2F cells independent of nutrient supply, as revealed by an immuno-gold assay.

Since other factors, such as the pro-apoptotic BH3-only protein BIM, have been shown to be regulated by a dynamic interaction with the dynein motor complex, via DLC122, it was investigated i) whether AMBRA1 is also part of this complex and, this being the case, ii) whether AMBRA1 association to this complex is modified during autophagy. To this end, coimmunoprecipitation experiments were performed in 2F cells, using an antibody specific for the dynein intermediate chain (DIC). It was observed that, under control conditions, AMBRA1 is associated with DIC and is therefore part of the wider dynein machinery (FIG. 2b, third lane). Moreover, upon autophagy induction obtained by nutrient starvation, AMBRA1 partially dissociated (65-70%) from the dynein complex (FIG. 2b, fourth lane), as also revealed by confocal immunofluorescence analysis. DIC immunodepletion experiments confirmed that AMBRA1 is still associated with DLC1 after being released from DIC. On the other hand, only a fraction of DLC1 interacts with AMBRA1, since most DLC1 is still present in the complex, where it plays alternative functions (FIG. 2b).

Importantly, the same interaction dynamics were also observed in kidney tissues from mice kept in the absence of food for 24-48 hours, thus emphasizing the physiological occurrence of this regulation (FIG. 2c).

Notably, AMBRA1 is also associated to the microtubule component Tubulin, from which it is released upon nutrient starvation (FIG. 2d). AMBRA1-Tubulin interaction is disrupted by mutating the DLC1 binding sites on the AMBRA1 moiety, confirming that AMBRA1's relationship with the cytoskeleton strictly depends on this accessory protein.

In order to investigate the upstream regulation of AMBRA1-DLC1 release from DIC, it was checked whether the serine/threonine kinase ULK1, whose yeast ortholog Atg1 is a key regulator of the pre-autophagosomal structure (PAS) formation, was essential for this dissociation upon autophagy induction. When ULK1 is down-regulated by small interfering RNA (siRNA) oligonucleotides, a remarkable reduction of the dissociation of the complex from DIC upon starvation conditions was observed (FIG. 2e) accompanied by autophagy impairment. Moreover, as revealed by western blot analysis of bidimensional gel electrophoresis, AMBRA1 is modified upon autophagy induction and this process is inhibited by ULK1 down-regulation. In line with this observation, it was found that AMBRA1 is phosphorylated by an ULK1 immunopurified complex (but not by its kinase-mutated form), as revealed by an in vitro kinase assay (FIG. 2f-g). By contrast, no modifications were detected by bidimensional gel electrophoresis for both DIC and DLC1. Consistent with this finding, inhibition of the c-Jun kinase (JNK), a crucial regulator of DLC1 phosphorylation during apoptosis induction, does not interfere with the dissociation process. Also, as expected, inhibition of BECLIN 1-VPS34 activity, a downstream event in autophagosome formation, does not interfere with the AMBRA1-DLC1 dissociation from DIC. Altogether, these data showed that AMBRA1-DLC1 dissociates from the dynein complex upon ULK1-dependent AMBRA1 phosphorylation.

It was then checked whether disruption of the microtubule network could interfere with the dynamics of AMBRA1 binding to the dynein motor complex. Nocodazole and Vinblastine, drugs which interfere with microtubule polymerisation and hinder autophagosome transport within the cytosol, did not impair AMBRA1-DIC interaction, suggesting that the association does not depend on microtubule integrity.

Interestingly, in parallel to the AMBRA1 dissociation from the dynein complex, autophagy induction led to an increase of AMBRA1 in the cell perinuclear region (FIG. 3a). It was therefore established which subcellular compartment AMBRA1 translocated to upon autophagy induction. By means of subcellular markers for endoplasmic reticulum (ER), autophagosomes, mitochondria, Golgi cisternae, early endosomes and lysosomes, a partial re-localisation of AMBRA1 was detected in the ER of 2F cells induced to autophagy by nutrient starvation (FIG. 3a). Notably, AMBRA1 mutants ^{TAT1} and ^{TAT2} are constitutively translocated to the ER, even in untreated conditions, confirming a role for DLC1 in regulating AMBRA1 dynamic localisation. Moreover, upon autophagy induction, a colocalisation of AMBRA1 with the FYVE protein DCFP1 was observed, which was recently described as a marker of the omegasome, a proposed site of autophagosome formation on the ER (FIG. 3b).

It was then verified whether the dynamic interaction of AMBRA1 with the dynein motor involved other components of the class III PI3-kinase complex3. Coimmunoprecipitation experiments showed that both VPS34 and BECLIN 1 are associated to AMBRA1 prior to and after autophagy stimulation (FIG. 3c, d). Consistent with this, the dynamic interaction of BECLIN 1 with DLC1 and DIC was also identified, similarly to AMBRA1 (FIG. 3d, e). Moreover, confocal analysis showed that BECLIN 1 follows the AMBRA1 translocation pattern after autophagy induction (FIG. 3f). Accordingly, VPS34 activity is also detectable at the site of translocation of AMBRA1 (FIG. 3g), as revealed by co-staining with the PI(3)P-interacting protein p40Phox. It should be noted that a fraction of BECLIN 1, restricted to the trans-Golgi network, does not co-localise with AMBRA1 (FIG. 3f), suggesting that two pools of BECLIN 1 may be differently regulated within the cell. Moreover, AMBRA1 down-regulation by RNA interference impairs BECLIN 1 translocation to the ER upon autophagy induction.

In the light of these results, it is proposed that, similarly to BIM regulation during apoptosis, the dynein motor complex might play a tethering/docking role in regulating autophagy by its association with AMBRA1 and the multi-molecular BECLIN 1-VPS34 autophagosome nucleation complex.

In order to elucidate the functional role of DLC1-AMBRA1 interaction in autophagy, it was tested whether DLC1 modulation resulted in autophagy dysregulation. To this end, DLC1 expression was down-regulated in 2F cells by transfection of DLC1-specific siRNA oligonucleotides (FIG. 4a) and then autophagy occurrence was analysed. Both conversion of LC3-I to its cleaved and lipidated form LC3-II and its translocation to autophagic structures—two sequential steps in autophagosome formation were assessed. Autophagy was also analysed by cell ultrastructural analysis and by measuring the increase in acidic vesicular organelles (AVOs). DLC1 down-regulation led to a remarkable increase in the number of autophagosome both in basal condition and during starvation—or Rapamycin-induced autophagy (FIG. 4b-d). This effect was also obtained in different cell types, such as neural precursor cells.

Dynein is composed of heavy and intermediated chain proteins involved in the structural composition of the motor complex in combination with different light chains which are known to modulate the complex's function. To verify whether autophagy was specifically induced by DLC1 down-regulation, autophagy occurrence was analysed in 2F cells transfected with siRNA oligonucleotides specific for other cytosolic members of the dynein light chain family, namely dynein TCTEX light chain 1, dynein ROADBLOCK light chain 1, and dynein light chain 2 (DLC2). Among these, only DLC2 down-regulation induced autophagy, although to a lesser extent than DLC1; accordingly, DLC2 was also able to bind AMBRA1.

Since it has been shown that the dynein complex is required for autophagosome migration and fusion to lysosome, it was investigated whether the accumulation of autophagosomes by DLC1 depletion could have derived from an enhanced autophagic sequestration (on-rate) or a reduced degradation of autophagic material (off-rate). To discriminate between these possibilities, the occurrence of autophagosomelysosome fusion was assessed in cells transfected with DLC1 siRNA oligonucleotides by monitoring the co-localisation of LC3 fused to mCherry (an improved-monomeric red-fluorescence protein that does not lose fluorescence under acidic condition, typical of lysosomes) with the lysosome markers LAMP1 and Lysotracker (not shown), in the presence of lysosomal protease inhibitors (E64d and pepstatin A), which prevent mCherryLC3 degradation within the lysosome. It was observed that, upon DLC1 down-regulation, autophagosomes are capable of fusing with lysosomes and forming autophagolysosomes (FIG. 4e). Moreover, E64d and pepstatin A enhanced the DLC1 siRNA-triggered increase of endogenous LC3-II (FIG. 4d) and the accumulation of GFP-LC3 puncta (FIG. 4f). Taken together these data imply that DLC1 inhibition increases the on-rate of autophagy in cultured cells.

Next, it was hypothesised that if autophagy induction by DLC1 down-regulation was due to the release of AMBRA1 from the dynein complex, this phenomenon should be abolished by AMBRA1 inactivation. 2F cells were transfected with DLC1 siRNA oligonucleotides alone or in combination with AMBRA1 siRNA oligonucleotides and analysed for the effect on autophagy by measuring the occurrence of GFP-LC3 puncta. As shown in FIG. 5a, autophagy induction by DLC1 down-regulation is prevented if AMBRA1 is concomitantly inactivated, indicating that DLC1′s role in autophagy is AMBRA1-dependent. Moreover, autophagy induced by DLC1 down-regulation requires BECLIN 1-VPS34 activity, as revealed by Wortmannin-mediated PI3K inhibition (FIG. 5b). To rule out the possibility that AMBRA1, besides its control of autophagy, could also have a role in the early endosome trafficking mediated by the dynein complex, the internalization and degradation of the EGF receptor was analysed in cells where AMBRA1 expression had been down-regulated by siRNA oligonucleotides. No significant differences were detected between AMBRA1 siRNA-transfected and control cells. Thus, AMBRA1 does not seem to play a role in early endosome trafficking.

Prompted by these data, it was decided to study the autophagic potential of AMBRA1 mutants lacking the DLC1 binding domain. 2F cells were transduced with retroviruses encoding the AMBRA1 wild type (AMBRA1 FL), or the AMBRA1^{TAT1} and ^{TAT2} mutants; autophagy induction was analysed by counting GFP-LC3-punctate positive cells or by measuring the increase in AVOs. Notably, AMBRA1 defective in DLC1-binding showed a stronger ability in inducing autophagy than did the wild type protein (FIG. 5c-d). These results support the hypothesis that AMBRA1 is bound in an inactive state to the dynein complex via the binding to DLC1 and that its release from the complex is an ULK1-dependent early step in AMBRA1-mediated autophagy.

DLC1 is a component of the microtubule-based molecular dynein motor complex. As such, it is involved in cell division, vesicular trafficking and ciliary/flagellar motility. However, DLC1 also interacts with proteins that are not directly associated with dynein- or microtubule-dependent roles, such as factors involved in apoptosis, enzyme regulation and viral pathogenesis. In particular, the pro-apoptotic BH3-only protein BIM is tethered by DLC1 on the dynein complex, from which is released upon induction of apoptosis by means of both DLC1 and BIM phosphorylation [triggered by p21-activated kinase 1 (Pak1) and Jun kinase (JNK), respectively]. Here it is shown that DLC1 may play also a role in regulating autophagy through its interaction with the pro-autophagic protein AMBRA1. In addition to its role in carrying the autophagosome towards the lysosome, our results imply an alternative, more regulative, role for the dynein motor complex in mediating autophagy initiation. Since microtubule dynamics are obviously linked to cell cycle regulation and cell motility, it could be speculated that AMBRA1 release from microtubules may also signal to the motor complex the initiation of the autophagy process, i.e. modulating the global citoskeleton response and rearrangement observed during autophagy.

Despite much progress in the field of autophagy, a critical question remains unanswered, i.e. how the autophagosomal membrane is formed. First, it is shown that ULK1 (an upstream regulator of autophagosome formation, involved in mAtg9 trafficking), may also act by regulating AMBRA1-DLC1 dissociation from the dynein complex. Second, it is reported that AMBRA1-DLC1, upon autophagy induction, translocates from the dynein complex to the ER. Autophagosome formation is known to require phosphatidylinositol-3-phosphate (PI3P) and is believed to occur near the ER50. Recent evidence points to the existence of a PI3Penriched membrane compartment, the omegasome, in dynamic equilibrium with the ER, which provides a platform for accumulation of autophagosomal proteins, expansion of autophagosomal membranes, and the emergence of fully formed autophagosomes.

Supporting this hypothesis is the fact that the anti-apoptotic factors BCL2 and BCLXL require ER localisation in order to regulate autophagy by BECLIN 1 binding.

It has been previously shown that AMBRA1 is required for an effective BECLIN 1-VPS34 complex formation. Now, our findings indicate that a pre-assembled complex, containing AMBRA1, BECLIN 1 and VPS34, translocates from the AMBRA1 docking site on the dynein motor complex to the omegasome compartment of the ER, where autophagosomes are initiated.

Autophagy is an evolutionary conserved process involved in a plethora of physiological and pathological processes. However, AMBRA1 has been identified as a vertebrate-specific gene. This implies that, either i) in lower eukaryotes other factors may play a function in the dynein-mediated control of autophagy, or ii) the regulation of autophagy by the dynein complex has evolved in vertebrates in order to achieve a fine tuning of the process in specific circumstances, such as mammalian embryogenesis.

Importantly, AMBRA1 is strongly expressed in adult brain compartments, such as hippocampus, cerebellum and striatum, which are all severely affected in neurodegenerative conditions. For this reason, the use of specific drugs able to deregulate AMBRA1-DLC1 interaction may prove highly useful in the therapeutic strategy to fight neurodegeneration.

In order to deregulate the AMBRA1-DLC1 interaction, three peptides (SEQ ID NO:1, SEQ ID NO:2 and SEQ ID NO:3) were selected for their likelihood to maintain the α-helix structure. SEQ ID NO:3 and SEQ ID NO:1 presented substitutions of the neutral residues of leucine into lysine, to increase the charge of the peptides (FIG. 5). The three peptides were synthesized in vitro with the addition of 11 arginine residues at the N-terminus and they were covalently conjugated with the FITC fluorochrome. The arginine residues allow an efficient incorporation of the peptides within the cells, whereas the FITC allows visualization of the peptides inside the cells and permit to monitor their stability upon time. Increasing doses of the peptides, respectively SEQ ID NO:3, SEQ ID NO:1 and SEQ ID NO:2 (100-200 μM) were added to the culture medium of HEK293 cells for increasing time (1-16 hours). The level of autophagy induced by the treatment was monitored by Western blot analyses of known markers of this process, such as LC3 and p62. It was observed that peptide with SEQ ID NO:1 induced an increase in autophagy as revealed by a decrease in p62 protein levels (FIG. 6A) and the increase in the shorter isoform of LC3 (data not shown). By contrast, peptide having SEQ ID NO:3 was not so effective at the doses used. Peptide having SEQ ID NO:2, identical to peptide having SEQ ID NO:1 but with the original leucine residues instead of lysine, gave similar results as peptide having SEQ ID NO:1 (data not shown). Densitometric analysis showed that the active peptides induced a 30-40% decrease in p62 protein (FIG. 6B), indicating an increase in autophagy. Although limited, this effect is somewhat more desirable than stronger effects, because it avoids massive autophagy that would likely results in cell death in vivo.

It is apparent to the person skilled in the art that modifications may be made to the methods and procedures without departing from the scope of the invention as set forth in the appended claims.

Experimental

Autophagy Assays

Autophagy was measured as described. In brief, starvation was induced by incubating cells in EBSS medium

(Sigma Aldrich) for 4-5 hrs. For immunodetection of LC3 puncta, cells were grown on coverslip and fixed with 4% paraformaldehyde in PBS, washed three times and directly examined by confocal microscopy. The results indicate the percentage of GFPLC3-positive cells with GFP-LC3 punctate dots or the numbers of GFP-LC3 punctate dots per cell. A minimum of 50-100 cells per sample were counted for triplicate samples per condition per experiment. To quantify the development of AVOs, cells were detached by trypsin digestion, washed with PBS, stained with acridine orange 1 pg/mL (Sigma-Aldrich) for 15 min and analysed using a FACScan flow cytometer (Becton Dickinson) and CellQuest software.

Autophagy studies in vivo were performed using C57BL/6 mice. Five month old male mice from the same littermate were kept without food but with water for 24 and 48 hours or fed ad libitum before sacrifice and tissue analysis.

For electron microscopy, cells were fixed with 2.5% glutaraldehyde in 0.1M cacodylate buffer pH 7.4 for 45 min at 4° C., rinsed in cacodylate buffer, postfixed in 1% OsO4 in cacodylate buffer, dehydrated and embedded in Epon. Ultrathin sections were briefly contrasted with uranyl acetate and photographed with a Zeiss CM900 electron microscope.

Antibodies

The primary antibodies used in this study were: rabbit anti-myc Tag antibody (Upstate Biotechnology), mouse anti-HA Tag antibody (Sigma-Aldrich), rabbit anti-DLC1 (Santa Cruz Biotech.), mouse anti-DIC (Santa Cruz Biotech.), rabbit and goat anti-BECLIN 1 (Santa Cruz Biotech., for IP and WB analyses, respectively), rabbit anti-VPS34 (Invitrogen), rabbit anti-LC3 (Cell Signaling), rabbit anti-AMBRA 1 (Strategic Diagnostic Inc., for WB analysis), rabbit anti-Ambra1 (Covalab, for IF and EM analysis), rabbit anti-Ambra1 CT (ProSci Inc., for IP and EM analysis), mouse anti-ERp57 (Stressgen), mouse anti-LAMP1 and anti-EEA1 (Abcam), mouse anti-GOLGIN (Invitrogen), mouse anti-Complex V α subunit (Invitrogen), mouse anti-β-tubulin (Sigma Aldrich), mouse anti-EGFR (Upstate Biotechnology), rabbit anti-Calreticulin (Stressgen).

Immunoprecipitation and Western Blot assays

In immunoprecipitation experiments, cells or tissues were lysed in HEMG buffer (25 mM Hepes pH 8.0, 100 mM NaCl, 25 mM MgCl₂, 0.5% Triton X-100, 0.1 mM EDTA 10% glycerol) plus protease and phosphatase inhibitors (Protease inhibitor cocktail plus 1mM Sodium Fluoride, 1 mM Sodium orthovanadate, 1 mM Sodium molibdate; Sigma-Aldrich). For analysis of the Beclin1-Dynein complex, immunoprecipitation was performed with a different lysis buffer (40 mM HEPES (pH7.4), 2 mM EDTA, 10 mM glycerophosphate, 0.3% CHAPS plus protease and phosphatase inhibitors) and a wash buffer (40 mM HEPES (pH7.4), 150 mM NaCl, 2 mM EDTA, 10 mM glycerophosphate, 0.3% CHAPS) as described in Hosokawa et al. 53. Lysates (1-3 mg) were then incubated at 4° C. for 30 min. Following a centrifugation at 4° C. for 10 min at 13000×g to remove insoluble debris, equal amounts of protein were incubated with 20 μl of monoclonal anti-cMyc or anti-FLAG antibody conjugated with protein A agarose beads (Clontech and Sigma-Aldrich, respectively) with rotation at 4° C. for 4 hr, or with 30 μl of monoclonal anti-HA antibody conjugated with protein A agarose (Sigma-Aldrich) or 2 μg of anti-DIC antibody overnight at 4° C., or 2 μg of anti-AMBRA1 antibody (CT from ProSci Inc.) followed by 60 min incubation with 30 μl of protein A/G sepharose beads (Roche). The beads were finally collected by centrifugation and washed four times with the HEMG buffer. Proteins bound to the beads were eluted with 50 μl of SDS-PAGE sample buffer and boiled at 95° C. for 10 min. In AMBRA-Tubulin interaction experiments, immunocomplexes were eluted by incubation with a FLAG peptide (Sigma-Aldrich) for 30 min at room temperature in order to prevent the presence of immunoglobulins in the gel.

Proteins were separated on NuPAGE Bis-Tris gel (Invitrogen) and electroblotted onto nitrocellulose (Protran, Schleicher & Schuell) or PVDF (Millipore) membranes. Blots were incubated with primary antibodies in 5% non-fat dry milk in TBS plus 0,1% Tween20 overnight at 4° C. Detection was achieved using horseradish peroxidase-conjugate secondary antibody (Biorad) and visualised with ECL plus (Amersham Bioscience). Note that endogenous AMBRA1 usually migrates as a doublet band, whereas the overexpressed AMBRA1 shows additional lower MW bands.

Confocal and Immunogold Analysis

For confocal analysis, cells were grown on coverslips and fixed with 4% PFA in PBS followed by permeabilisation with 0.1% triton X-100 in PBS. Primary antibodies were incubated for 1 hour at room temperature and visualised by means of Cy3- and Cy2-conjugated secondary antibodies. (Jackson Immuno Research). Coverslips were mounted in SlowFade-Anti-Fade (Invitrogen) and examined under a confocal microscope (Leica TCS SP2).

For electron microscopy, 2F cells were fixed in 2% freshly depolymerised paraformaldehyde and 0.2% glutaraldehyde in 0.1M cacodilate buffer pH 7.4 for 1 hour at 4° C. Samples were rinsed in buffer, partially dehydrated and embedded in London Resin White (LR White, Agar Scientific Ltd.). Ultrathin sections were processed for immunogold technique. Grids were pre-incubated with 10% normal goat serum in 10 mM PBS containing 1% bovine serum albumine (BSA) and 0.13% NaN3 (medium A), for 15 minutes at RT; sections were then incubated with primary antibody, rabbit polyclonal anti AMBRA1 (Covalab or ProSci Inc.) diluted 1:50 in medium A, overnight at 4° C. After rinsing in medium A containing 0.01% Tween 20 (Merck), sections were incubated in goat anti-rabbit IgG conjugated to 15 nm colloidal gold (British BioCell Int.), diluted 1:30 in medium A, containing fish gelatine, for 1 hour at RT. After incubation in medium A for 15 minutes at RT, a second immunolabelling was performed utilising rabbit polyclonal anti-DLC1 (Santa Cruz) as primary antibody and goat anti-rabbit IgG conjugated to 5 nm colloidal gold as secondary antibody.

Grids were thoroughly rinsed in distilled water, contrasted with aqueous 2% uranyl acetate for 20 minutes, and photographed in a Zeiss EM 900 electron microscope.

Statistical Analysis

Microsoft Excel was used for statistical analysis. Statistical significance was determined using the Student's t-test. A P value of equal to or less than 0.05 was considered significant.

1.-12. (canceled)

13. A peptide comprising a TQT amino acid triplet followed by at least 5 amino acid residues forming an u-helix secondary structure.

14. A peptide according to claim 13, characterised by comprising a sequence having at least 90% identity with SEQ ID NO: 1 or SEQ ID NO: 2 or SEQ ID NO: 3.

15. A peptide according to claim 13, characterised by comprising asequence having SEQ ID NO: 1 or SEQ ID NO: 2 or SEQ ID NO: 3.

16. A peptide according to claim 13, characterised by being SEQ ID NO: 1 or SEQ ID NO: 2 or SEQ ID NO: 3

17. A peptide according to claim 13 for use as a medicament.

18. A pharmaceutical composition containing at least one peptide according to claim 13 in mixture with at least one pharmaceutically acceptable vehicle and/or excipient.

19. A method of preventing binding of Ambra1 to DLC1 by interfering with their reciprocal interaction comprising use of a peptide according to claim 13.

20. A method of treating diseases deriving from the dysregulation of the signalling system of Ambra1-mediated autophagy comprising use of a peptide according to claim 13.

21. The method according to claim 20, characterised in that such diseases are neurodegenerative diseases or oncogenesis.

22. The method according to claim 21, characterised in that said neurodegenerative disease is selected from the group consisting of Alzheimer's disease, Huntington's disease and Batten disease.

23. A peptide according to claim 13 for use in treatment of neurodegenerative diseases or tumorigenesis.

24. A peptidomimetic compound comprising at least one portion having the same 3D conformation of a peptide according to claim 13.

25. A peptidomimetic compound according to claim 24 for use as a medicament.

26. A pharmaceutical composition containing at least one peptidomimetic compound according to claim 24 in mixture with at least one pharmaceutically acceptable vehicle and/or excipient.

27. A method of preventing the binding of Ambra1 to DLC1 by interferin with their reciprocal interaction comprising use of a peptidomimetic compound according to claim 24.

28. A method of treating diseases deriving from the dysregulation of the signalling system of Ambra1-mediated autophagy comprising use of a peptidomimetic compound according to claim 24.

29. The method according to claim 28, characterised in that such diseases are neurodegenerative diseases or oncogenesis.

30. The method according to claim 29, characterised in that said neurodegenerative disease is selected from the group consisting of Alzheimer's disease, Huntington's disease and Batten disease.

31. A peptidomimetic compound according to claim 24 for use in treatment of neurodegenerative diseases or tumorigenesis.

32. A method of preventing the binding of Ambra1 to DLC1 by interfering with their reciprocal interaction comprising use of a composition according to claim 18.

33. A method of treating diseases deriving from the dysregulation of the signalling system of Ambra1-mediated autophagy comprising use of a composition according to claim 18.

34. The method according to claim 33, characterised in that such diseases are neurodegenerative diseases or oncogenesis.

35. The method according to claim 34, characterised in that said neurodegenerative disease is selected from the group consisting of Alzheimer's disease, Huntington's disease and Batten disease.

36. A composition according to claim 18 for use in treatment of neurodegenerative diseases or tumorigenesis.

37. A method of treating preventing the binding of Ambra1 to DLC1 by interfering with their reciprocal interaction comprising use of a composition according to claim 26.

38. A method of treating diseases deriving from the dysregulation of the signalling system of Ambra1-mediated autophagy comprising use of a composition according to claim 26.

39. The method according to claim 38, characterised in that such diseases are neurodegenerative diseases or oncogenesis.

40. The method according to claim 39, characterised in that said neurodegenerative disease is selected from the group consisting of Alzheimer's disease, Huntington's disease and Batten disease.

41. A composition according to claim 26 for use in treatment of neurodegenerative diseases or tumorigenesis.


SEQUENCE LISTING (3 sequences)

SEQ ID NO: 1 - protein, 11 aa, Homo sapiens
SQGTQTKAKQK

SEQ ID NO: 2 - protein, 11 aa, homo sapiens
SQGTQTLALQL

SEQ ID NO: 3 - protein, 17 aa, Homo sapiens
TQTKAKQKQNAETQTER